(19) United States
(12) Patent Application Publication
(10) Pub. No.: US 2023/0148957 A1
(43) Pub. Date: May 18, 2023

(54) A METHOD AND A SYSTEM FOR DETECTING AND OVERCOMING A DEVIATING GAIT

(71) Applicant: CERITER, Lanaken (BE)

(72) Inventor: Piet STEVENS, Maastricht (NL)

(21) Appl. No.: 17/917,082

(22) PCT Filed: Apr. 2, 2021
(86) PCT No.: PCT/NL2021/050219

§ 371 Date: Oct. 5, 2022

(30) Foreign Application Priority Data
Apr. 5, 2020 (BE) 2020/0034

(51) Int. Cl. A61B 5/00; A61B 5/103; G16H 10/60; G16H 40/67

(52) U.S. Cl. CPC A61B 5/486; A61B 5/1038; A61B 5/6807; A61B 5/7264; A61B 5/7246; A61B 5/0022; G16H 10/60; G16H 40/67; A61B 2562/0247; A61B 2562/04; A61B 5/4082

(57) ABSTRACT

A method for detecting and alleviating symptoms of a deviating gait disorder in a human patient, includes the steps of: a) continuous registering a person's gait using a sole embedded in the shoes of the patient, the sole comprises at least two pressure sensors in or on the surface of the sole, b) sending information based on a person's gait from the sole to a receiver device, c) analyzing and interpreting the information received by the receiver device in order to detect whether the gait is deviating or not, d) upon detecting a deviating gait, sending single or multiple cues to the patient in order to overcome the deviating gait.

### FIELD OF THE INVENTION

The present invention relates to a method and a system for detecting and overcoming a deviating gait.

### BACKGROUND

Parkinson's disease (PD), or simply Parkinson's, is a long-term degenerative disorder of the central nervous system that mainly affects the motor system. As the disease worsens, non-motor symptoms become more common. The symptoms usually emerge slowly. Early in the disease, the most obvious symptoms are shaking, rigidity, slowness of movement, and difficulty with walking. Thinking and behavioral problems may also occur.

Festinating gait is a typical gait exhibited by patients suffering from PD. It is a form of involuntary acceleration, manifested by short shuffling steps. By making shorter and shorter steps, the feet stay behind and the body tilts forward. This creates an increased risk of falling. Freezing of Gait (FoG) is another symptom of PD, and is defined as “a brief episodic absence or marked reduction of forward progression of the feet despite the intention to walk”, and is an important contributor to falls, leading to it being a major cause of hospitalization.

Cueing is a well-established technique that has been shown to improve gait in people with Parkinson's, including increasing walking speed, step length, cadence and reducing the number of FoG episodes.

Three cueing modalities are extensively reported in literature, visual cueing, auditory cueing and somatosensory cueing. An example of visual cueing is spaced parallel lines drawn on the floor, over which the patient should step in order to regulate their step length. An example of auditory cueing is a repeated audible sound, such as the ticking sound of a metronome, which can be followed to step in time to the beat of the metronome. Shoulder tapping is an example of a somatosensory stimulus, the pace of which can be followed by the patient.

Sweeney et al. 2019, describes a wired headphone around the neck of a user connected to a wearable computer attached to the lower back, and an acceleration sensor attached to the shin and connected to the wearable computer over a Bluetooth classic communication link. The document further describes a similar system comprising a wireless ear-device instead of a wired headphone, a sensor unit around the user's waist and a smartphone with auditory cueing application worn on-body. A third similar system is described comprising (wired) earphones, two sensor units attached to each lower leg (or ankle) and a smartphone with auditory cueing application worn on-body.

These known devices and/or methods are not really user-friendly or fit for daily use, for example when they require sensors to be attached to ankles, requiring the help of another person or specific calibration walks to set up algorithms for high accuracy and sensitivity recognition. In addition, they are not sensitive or flexible enough to provide an adequate real-time detection of deviating gait and as a consequence, the cueing will be impaired as well. Also, the detection devices and/or methods of the prior art do not differentiate/distinguish between different types of deviating gait (such as festination of freezing) or circumstances (freeze during turn vs freeze during straight walking).

Accordingly, a need arises for an improved method and system for detecting and overcoming a deviating gait. The current invention aims to provide a solution for at least one of the problems mentioned above.

### SUMMARY OF THE INVENTION

The present invention and embodiments thereof serve to provide a solution to one or more of above-mentioned disadvantages. To this end, the present invention relates to a method according to claim 1. Embodiments of the method are provided in any of the dependent method claims. The method claims disclosed in the set of claims as filed are incorporated by reference in this description in their entirety.

In a second aspect, the present invention relates to a system according to claim 22. Embodiments of the system are disclosed in any of the dependent system claims. The system claims disclosed in the set of claims as filed are incorporated by reference in this description in their entirety.

Both method and system are more sensitive, adapt better to the specific circumstances, user-friendly and/or effective to reduce deviating gaits and therefore are more reliable than solutions hitherto known in the art.

### DESCRIPTION OF FIGURES

FIG. 1 shows a schematic representation of the system with insole pressure measurement (100), the smartphone and app (101), and auditory and haptic cueing (102) and cloud portal (104). All system components are shown together with the user (103), the care professional (105) and all data flows according to an embodiment.

FIG. 2 shows the system in use with all system components highlighted (the pressure sensor insole (205), the smartphone (203), the in-ear headphone (201—one-sided), an optional button (200) for the user to provide input to the system and the interface to the portal (202) according to an embodiment.

FIG. 3 shows the pressure measurements for a healthy person walking with clear heel down (300) and toe off (301) pressure data according to an embodiment. The horizontal axis is time and the vertical axis is a measure of pressure.

FIG. 4 shows the legend for FIGS. 3 and 5. The inside heel sensor (“heel in”) has a line style of “long dash dot”. The outside heel sensor (“heel out”) has a line style of “long dash dot dot”. The main measurements of concern for this invention are the hallux (continuous line), the toes (dots only) as well as the 2 signals from the heel (heel in and heel out).

FIG. 5 shows the pressure measurements for a person with Parkinson going from festination (lack of normal rolling off of the foot, clear heel down, minimal toe off (500)) to no toe off (501) to FOG (person in this case on heels which no longer leave the floor (502)) according to an embodiment. The horizontal axis is time and the vertical axis is a measure of pressure (pressure increases in the direction of the arrow).

FIG. 6 shows the wireless recharging mechanism for the transmitter according to an embodiment.

In the context of the present disclosure, “heel down” means high pressure for both sensors under the heel (“heel in” and “heel out”).

In the context of the present disclosure, “toe off” means high pressure for both sensors under the toes (“hallux” and “toes”).

In the context of the present disclosure, “high pressure” means pressure as indicated in the graph close to the top end of the vertical axis.

### DETAILED DESCRIPTION OF THE INVENTION

The present disclosure concerns a method and a system for detecting and overcoming a deviating gait.

Unless otherwise defined, all terms used in disclosing the method and/or the system as claimed, including technical and scientific terms, have the meaning as commonly understood by one of ordinary skill in the art. By means of further guidance, term definitions are included to better appreciate the teaching of the present disclosure.

As used herein, the following terms have the following meanings:

“A”, “an”, and “the” as used herein refer to both singular and plural referents unless the context clearly dictates otherwise. By way of example, “a sensor” refers to one or more than one sensor.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

The terms or definitions used herein are provided solely to aid in the understanding of the disclosure.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the present disclosure relates to a method comprising steps of:
a) continuously registering a person's gait using a sole embedded in the shoes of said patient, said sole comprises at least two pressure sensors in or on the surface of said sole,
b) sending information based on a person's gait from said sole to a receiver device via a transmitter,
c) analyzing and interpreting said information received by said receiver device in order to detect whether said gait is deviating or not,
d) upon detecting a deviating gait, sending single or multiple cues to said patient in order to overcome said deviating gait.
e) upon detecting non-deviating gait, optionally sending single or multiple cues to said patient in order to reinforce non-deviating gait.

In the context of present disclosure, the term “deviating gait disorder” means any disorder that produces a deviation from the gait of a healthy person that can imperil the person with the deviating gait disorder.

In the context of the present disclosure, a “pressure sensor” is an electronic device that will generate an electric signal that is proportional to the pressure exerted on the surface of the device.

In the context of the present disclosure, a “transmitter” is an electronic device that can receive information from the sole with pressure sensors and can wirelessly transmit this information.

In the context of the present disclosure, a “receiver device” (short: receiver) is any electronic device that can receive gait information, for example from pressure sensors, and which can apply a stored algorithm to that gait information in order to detect gait deviation and generate cues.

In the context of the present disclosure, “detecting gait deviation” means categorizing gait at every point of time as either normal or deviating and also classifying deviating gait in different categories, for example festination or freezing.

In the context of the present disclosure, a “cue” is any signal sent to the user of the method or the system that allows the user to adapt their behavior in some form.

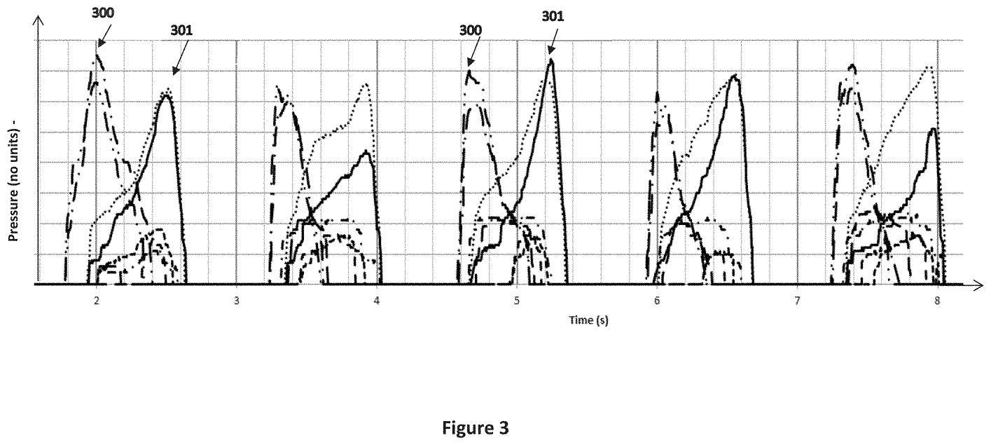

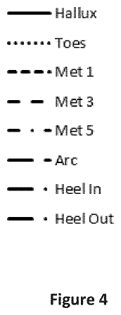

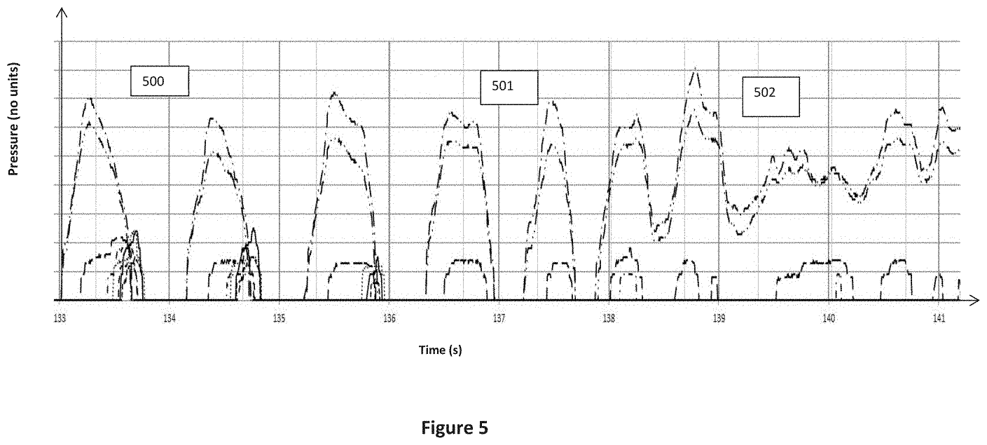

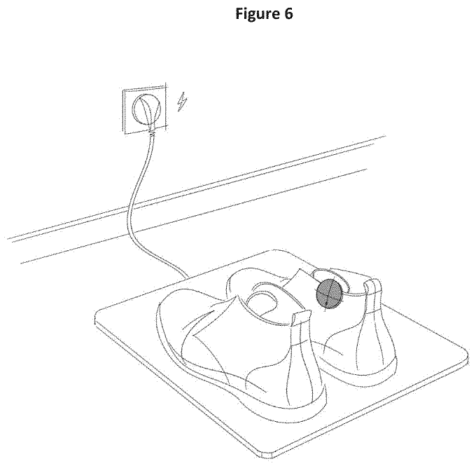

In order to illustrate “deviating gait”, reference is made to FIGS. 3 and 5. FIG. 3 shows a healthy person's gait with clearly visible heel down (300) and toe off (301) phases of the gait. FIG. 5 shows two aspects of a deviating gait disorder: festination and freezing of gait.

Comparing the first 2-3 steps (500) of this recording to the steps of a healthy patient in FIG. 3, one notices that the toe off is much less pronounced for this patient. For festination (501), one can see that there is no real toe off in the pressure pattern and the slope of the pressure increase in heel down is also less than for a healthy patient. The rightmost part of the figure shows freezing of gait (502) whereby one can see that the heels touch the floor continuously and the toes barely touch the floor or not at all. There are also patterns of freeze where the toes are on the floor and the heels are not.

In a preferred embodiment, said pressure sensors detect pressure variations of the foot on said sole when walking.

In a preferred embodiment, said pressure sensors are integrated in an sole which is inserted in the shoe.

Preferably, said sole comprises at least five pressure sensors, preferably located under the heels, under the toes and in metatarsal positions.

In an embodiment, said pressure sensors are connected to said transmitter that will wireles sly transmit gait information from the pressure sensors.

In an embodiment, an optional feedback box adds the possibility to provide feedback from the user to the app in the smartphone.

In an embodiment, said information from said pressure sensors arrives at said receiver, wherein said information is analyzed by a computerized algorithm in order to detect whether said gait is deviating or not.

In a further embodiment, said computerized algorithm is stored in the cloud (for example in a cloud based device communicating with the receiver device), on the smartphone or on said receiver.

In a further embodiment, said computerized algorithm analyses detailed timing information of said sensors, accumulating averages during normal gait and identifying deviations from those averages per area of the foot, such as toes, heels and metatarsals to detect whether said gait is deviating or not and in which way it is deviating. In a further aspect, said normal gait is based on an average of at least two or three steps. A first advantage of using an average pressure parameter of a number of steps for normal gait to determine a deviating gait by the computerized algorithm, is that the risk of sending a cue based on a misinterpretation by the computerized algorithm of a single step during walking is reduced. A second advantage of using an average pressure pattern of a number of steps for normal gait to determine a deviating gait by the computerized algorithm is that the method recognizes external variations not related to the person whose gait is being registered. For example, an external variation can be a change of surface, for example from a paved surface to grass, over which the person walks. By taking the average of at least two previous steps as normal gait, an automatic variable adjustment to the pattern of the normal gait is applied in a dynamic/adaptive way. In this way, the risk is reduced that the method misinterprets information regarding a next step registered by the pressure sensors of the sole as a result of external factors not related to the person, i.e. information of the next step registered by the sensors is analyzed or compared by the computerized algorithm with an average of a number of previous steps. Hence, by using a variable normal gait over time, based on an average of at least two or three previous steps, by the computerized algorithm (or the device of the system as discussed below) the accuracy of the method (or system) disclosed in this disclosure is significantly increased.

In a further embodiment, said computerized algorithm analyzes heel down and toe off pressures to detect whether gait is deviating or not and in which way it is deviating. The heel down and toe off pressures to be analyzed by said computerized algorithm provide a reliable and/or efficient determination of a deviating gait.

In another aspect of the method, the computerized algorithm monitors, after providing the single cue or multiple cues, in real time the person's gait for determining a deviating gait reduction, wherein by determining by the computerized algorithm in real time that the deviating gait reduction is below a threshold a second single cue or second multiple cues is/are provided, wherein the previous single cue or previous multiple cues is/are different than the second single cue or second multiple cues, for example by a different type of cuing, different duration of cuing, different frequency of cueing, different volume of cuing, different tactile/visual experience of cuing or different auditory instructions of cuing or by using two cueing devices simultaneously. By instantaneous determination of a deviating gait reduction by the computerized algorithm, and by using a different cue or cues than the first cue(s) after determination of a deviating gait reduction below a threshold, the effectiveness of the method may be increased significantly, in particular for persons having PD. This process may be repeated for subsequent cues and/or these different cues may be customized for each user and/or customized to the specific deviating gaits of each user. The threshold may also be customized for each user and/or the threshold is a variable threshold depending on the recognized deviating gait pattern (freezing/festination, heel down, toes down, trembling, akinesia, etc.). In this manner it is possible to recognize a deviating pattern and to apply a dynamic interaction between cueing and deviating gait pattern, which may be based on user customized cueing strategies, such that the user returns to normal gait in a relatively fast manner.

The computerized algorithm is analyzing said information registered by the pressure sensors of the sole by comparing said information with a normal gait pressure pattern. Said normal gait pressure pattern may be based on pressure patterns of at least two or three previous steps of the registered person's gait. In another aspect, said normal gait pressure pattern is based on an average of at least two or three previous steps of the registered person's gait. By using the pressure patterns of two or three steps of the registered person's gait and/or the average pressure pattern of at least two or three steps for a normal gait pressure pattern used by the computerized algorithm to determine deviating gait in a subsequent step, the accuracy of the method may be significantly increased. Hence, the threshold used by the computerized algorithm can be based on certain averages of previous steps. So, the threshold does not change in a fixed manner from one predetermined deviation threshold to another predetermined deviation threshold, but it starts from an initial threshold which is then continuously modified, e.g. by having a running average for normal gait or the normal gait pressure pattern used by the computerized algorithm to determine deviating gait.

In a different aspect, if said deviating gait is determined by the computerized algorithm, deviating gait can be overcome in a more effective manner by changing the mode used by the computerized algorithm from a first mode used for normal gait to a second mode after determining deviating gait, and/or vice versa after determining normal gait for a predetermined time period after determining deviating gait. In the second mode, pressure or pressure patterns registered by the sensors of the sole are associated to specific cues. So, after identifying deviating gait, or a deviating gait category such as freezing or festination, the method is sending a predetermined cue associated to the registered pressure or the registered pressure pattern, wherein each registered pressure or each registered pressure pattern to be detected by computerized algorithm may have an associated (specific) cue to increase the effectiveness of cueing to overcome deviating gait. The associated cue or cues to be sent may be customized to the user.

As shown in the FIG. 5, freezing or festination have different pressure patterns, wherein different cues can be associated to freezing compared to festination to overcome festination or freezing in a relatively fast manner, for example by using customized cues or cues particularly helpful to overcome freezing or other cues (more) effective to overcome festination. Also during freezing different specific cues can be used for each different registered pressure to increase the effectiveness of cueing to overcome deviating gait.

In a further embodiment, said ways of deviating may be classified into festination and FOG (separately).

In an embodiment, said one or more cues are sent within 1 second after detecting said deviating gait.

In a further embodiment, said gait deviations may influence number and type of said cues in order to provide cues that have real impact on the user.

In a further embodiment, the said number and type of said cues may also be influenced by pre-set parameters in the algorithm.

In a further embodiment, cues may also be generated from time to time during normal gait.

In an embodiment, said receiver device is a wearable computer device, preferably a smartphone.

In an embodiment, said cue is an auditory, haptic and/or visual cue.

In an embodiment, said cues are received by a cueing device, said cueing device comprises a headphone, a vibrating device and/or a projection/light emitting device. The vibrating or haptic device may be releasably attached by a strap or the like to the person's upper body around or above the person's waist such as around an arm. Attaching a vibrating or a haptic device around or above the person's waist increases the responsiveness of the person compared to attaching a vibrating or a haptic device below the person's waist, such as to the legs or feet.

Said cueing device and receiver may be present in one device.

In an embodiment, said cueing device and receiver device are present in multiple separate devices that can communicate with one another. In addition, the sole comprising the pressure sensors may be another device communicating in a wireless manner with the cueing device by means of the receiver device, such that in an user-friendly way an optimal response of the person to a cue of the cueing device may be obtained to overcome deviating gait.

In an embodiment, information from a person's gait is collected in a database, preferably a cloud database.

In the context of the current disclosure, a database is a method for storing and retrieving data, typically running on a server.

In the context of the current disclosure, a server is a computer with enough processing power and storage to accommodate said database and with communication facilities to allow easy access for storage and retrieval of said data. In addition, said server may run a number of applications to analyze the data.

In the context of the current disclosure, the cloud is a set of said servers which may be fully or partially rented or wholly owned by the person or organization running said database and said applications on said servers.

In the context of the current disclosure, a cloud database is said database running in said cloud.

In the context of the current disclosure, a portal is an application that gives access to said database in such a way that the user is represented with a report in tabular (tables) or graphic format (graphs) that allows said user to interpret the data and draw conclusions from it.

In the context of the current disclosure, a cloud portal is a portal that runs in said cloud.

In a further embodiment, the said user feedback is sent to said database.

In a further embodiment, said cloud database is analyzed by special software across patients in order to group patients according to personal or gait characteristics.

In a further embodiment, said gait data is analyzed and quality of gait indicators are computed.

In the context of the current disclosure, quality of gait indicators are numeric indicators of the quality of gait, for example, the ratio of the amplitude of the pressure peaks to the pressure trough, the ratio of the amplitude of the heel down pressure to the toe up pressure or the average number of peaks per foot down over time.

In an embodiment, said human patient suffers from Parkinson's Disease.

In a further embodiment, said human patient suffers from CVA.

In a further embodiment, said human patient suffers from MS.

In a further embodiment, said human patient suffers from other neurological diseases impacting gait, such as poly-neuropathy.

In a further embodiment, said human patient suffers from amputations of the lower limbs.

In an embodiment, said symptoms are festinating gait or freezing of gait.

In a further embodiment, said symptoms are circumduction.

In a further embodiment, said symptoms are phantom pains.

In a second aspect, the present disclosure relates to a system comprising:
a) a sole comprising at least two pressure sensors in or on the surface of said sole for registering a person's gait,
b) a sender sending information based on a person's gait from said sole to a receiver device,
c) a receiver device, for receiving said information,
d) a device for analyzing and interpreting said received information in order to detect whether said gait is deviating or not,
e) a cueing device, sending single or multiple cues to said patient in order to overcome said deviating gait.

The system of present disclosure can further comprise:

a cloud portal giving a care professional access to patient data, and/or
a cloud portal analyzing algorithm performance over time and modifying the algorithm in the receiver in order to enhance algorithm performance,
and/or a cloud portal giving a patient or their care givers access to gait performance.

A person of ordinary skill in the art will appreciate that the system of this second aspect can be used in the method as described above. Consequently, all aspects of the present disclosure are related. All features as described in the aspect concerning the method or in the aspect concerning the system, as described above as well as below, can relate to any of these aspects, even if they are described in conjunction with a specific aspect. Hence, a device for analyzing and interpreting said received information in order to detect whether said gait is deviating or not as defined in system claim 1, is a device on which the computerized algorithm (as disclosed above for the method) runs. So, the device is configured for determining that said person's gait is deviating by analyzing information based on a person's gait obtained by pressure sensors provided in a sole. The device is communicating, for example in a wireless manner, with a cueing device for sending at least one cue in order to overcome a determined deviating gait. The device and the receiver device may be present in one device or the device of the system is a cloud based device, wherein the cloud based device communicates with the receiver device

In the context of the current disclosure, a care professional is a person with a medical education such as neurologist, revalidation specialist, medical doctor, physiotherapist or occupational therapist.

In the context of the current disclosure, access to patient data means that a care professional receives a report representing measured patient data from said sole or said accelerometer in tabular or graphic format so that the care professional can assess the development of the disease, the appropriateness of the medication or its dosage or the appropriateness of any other intervention to improve the patient's health.

In the context of the current disclosure, algorithm performance means the quality of recognition of deviating gait (such as F1-score or sensitivity) and the quality of the cueing (in terms of bringing the deviation back to zero, reducing the number of episodes of festination or freezing and/or their length).

In the context of this disclosure, the patient's gait performance is the percentage of time the patient walks normally as a percentage of the total time walked.

In an embodiment, relationships between groups of patients, quality of gait indicators and algorithm parameters are analyzed, leading to improved algorithm performance and adaptation of the algorithm to the evolution of the disease in individual patients.

In the context of the current disclosure, algorithm parameters mean the input parameters that can adapt the generic algorithm to a specific patient. An example of said algorithm parameters are the percent deviation from normal foot down time before such a deviation is interpreted as freeze of gait. Another example is the threshold ratio of the heel down pressure to the pressure trough in the middle of foot down that indicates start of festination.

In a preferred embodiment, said sole further comprises at least one accelerometer, magnetometer or gyroscope.

In a further embodiment, said accelerometer may be included in said transmitter.

In an embodiment, said sole comprises at least five pressure sensors, preferably located under the heels, under the toes and in metatarsal positions.

In an embodiment, said receiver device is a wearable computer device, preferably a smartphone.

In an embodiment, said cueing device is configured to provide an auditory, sensory (haptic) and/or visual cue.

In a further embodiment, in addition to the pressure data analysis described above, said computerized algorithm analyses said accelerometer data, optionally in combination with pressure data, to identify and classify deviating gait patterns.

In an embodiment, said computerized algorithm generates cues specific to certain movements, such as for example a sharp turn.

In a further embodiment, said computerized algorithm generates different cues for different classes of deviating gait.

In an embodiment, said cues are sent by a cueing device, said cueing device comprises a headphone, a vibrating device and/or a projection/light emitting device. The vibrating device may be releasably attached by a strap or the like to the person's upper body around or above the person's waist such as around an arm to increase the responsiveness of the person to a vibrating device.

In the context of this disclosure, cueing parameters are the inputs to the algorithm that define the type, duration and frequency of cueing as well as the circumstances in which each type of cue must be used. For example, using a metronome sound (vs recorded voice) would be one cueing parameter. Which type of cue to use in a sharp turn would be another example of a cueing parameter. Which voice and which message would be yet another example of cueing parameters for recorded voice.

In an embodiment said cueing device and receiver are present in one device.

Said cueing device and receiver may be present in multiple separate devices that can communicate with one another and with the receiver. In an embodiment of the system, the cueing device and receiver device are present in multiple, communicating devices. In addition, the sole comprising the pressure sensors is preferably another device communicating in a wireless manner with the cueing device by means of the receiver device.

In another embodiment, the device is configured to analyze heel down and toe off pressures to detect whether gait is deviating or not and/or in which way it is deviating. Heel down and toe off pressures provide a reliable and/or efficient determination of a deviating gait.

In a further embodiment, after providing the single cue or multiple cues, the device is configured to monitor in real time the person's gait for determining a deviating gait reduction, wherein by determining in real time that the deviating gait reduction is below a threshold a second single cue or second multiple cues is/are provided, wherein the previous single cue or previous multiple cues is/are different than the second single cue or second multiple cues, for example by a different type of cuing, different duration of cuing, different frequency of cueing, different volume of cuing, different tactile/visual experience of cuing or different auditory instructions of cuing or by using two cueing devices simultaneously. By instantaneous determination of deviating gait reduction by the device, and by using a different cue or cues than the first cue(s) after determination of a deviating gait reduction below a threshold, the effectiveness of the system may be increased significantly, in particular for persons having PD.

Further, the device may be configured to analyze said information by comparing said information with a normal gait pressure pattern. Further, the device is configured to base said normal gait pressure pattern on pressure patterns of at least two or three previous steps of the registered person's gait. In a further aspect, the device is configured to base said normal gait pressure pattern on an average of at least two or three previous steps of the registered person's gait. By using the pressure patterns of two or three steps of the registered person's gait and/or the average pressure pattern of at least two or three steps for a normal gait pressure pattern to determine deviating gait in a subsequent step, the accuracy of the system may be significantly increased.

The device may be configured to determine deviating gait above a predetermined deviation threshold. Further, the device is configured to change from a first mode used for normal gait to a second mode to overcome deviating gait, and/or vice versa after determining normal gait for a predetermined time period after determining deviating gait, wherein in the second mode, pressure or pressure patterns registered by the sensors of the sole are associated to specific cues. So, after identifying deviating gait, or a deviating gait category such as freezing or festination, the cuing device is sending a predetermined cue associated to the registered pressure or the registered pressure pattern, wherein each registered pressure or each registered pressure pattern to be detected by the device may have an associated (specific) cue to increase the effectiveness of cueing to overcome deviating gait. The associated cue or cues to be sent may be customized to the user.

After identifying deviating gait, or a deviating gait category such as freezing or festination by the device, the cuing device is configured to send at least one predetermined cue associated to the registered pressure or the registered pressure pattern, wherein each registered pressure or each registered pressure pattern to be detected by the device has an associated (specific) cue to be provided by the cuing device. In this way, the system overcomes a deviating gait in a relatively fast and effective manner.

In an embodiment, the portal presents to the care professional a report indicating the frequency of deviating gait over time (for example, the number of festination or freezing episodes per day).

In an embodiment, the portal presents the care professional a report indicating the patient manual feedback over time (from said button).

In an embodiment, the portal calculates algorithm performance indicators over time and analyzes how they would be improved by varying the algorithm parameters in order to obtain better deviating gait recognition.

In the context of this disclosure, the cloud software that analyses algorithm and cueing performance is called the analysis software.

In an embodiment, the portal varies the cueing parameters the patients receive in order to determine which cueing parameters generate the best patient response in terms of bringing deviating gait back to normal or reducing it (either in frequency and/or duration).

In an embodiment, the portal groups patients based on their characteristics (such as cueing parameters or algorithm parameters but also other data such as age, period of time since diagnosis etc . . . ) and accelerates optimization of algorithm performance and cueing effect by applying lessons from one patient to the entire group.

In an embodiment, the portal provides feedback to the patient and their caregivers regarding their gait performance and obtains feedback on overall system effectiveness in order to use this to improve said system effectiveness.

In the context of this disclosure, system effectiveness is the overall effect of all system components (insole, accelerometer, transmitter, receiver, cueing mechanism, input button and portal) on the patient's gait performance.

The disclosure is further described by the following non-limiting examples and/or figures which further illustrate the invention, and are not intended to, nor should they be interpreted to, limit the scope of the invention.

### EXAMPLES

### Example 1: Setup before First Use by One Patient

Before first use, the patient and/or a care giver go through a setup dialog to indicate starting parameters. They indicate what type of cueing works for the patient, for example auditory cueing with the voice of the patient's physiotherapist counting from one to two and with a special message in case the patient experiences a long episode of freezing and which has been shown to allow him to get out of the freeze. They also decide that they do not want the system or method to generate positive cueing when the patient walks without problems, but that they do want the analysis software in the portal to optimize algorithm and cueing parameters without them having to manually approve such changes each time they are introduced.

As suggested by the app, they leave the other parameters, such as the minimum number of steps taken before cueing starts at their default values.

### Example 2: Use by One Patient During Walking

To better exemplify reference is made to FIG. 2, which show(s) the patient using the system and method while walking, FIG. 3 which shows a healthy gait pattern, FIG. 5 which shows a deviating gait pattern and FIG. 6, which shows the recharging of the transmitter.

A patient will charge the transmitter on a regular basis (for example once per week) by placing the shoes on which the transmitter has been clipped, on a recharging mat as per FIG. 6.

FIG. 2 shows a patient walking with an insole with pressure sensors in his left shoe. The insole is connected to a transmitter clipped on the patient's shoe, which transmits pressure data to a smartphone in the patient's pocket which runs an app analysing the pressure data.

As the patient walks normally, the app receives and records pressure data as illustrated in FIG. 3, which it categorizes in real-time as normal gait. As the patient has indicated no cueing is necessary during normal gait, the algorithm provides no cueing.

The patient approaches an open door and the gait pattern starts to modify as per FIG. 5 (500). As the toe up decreases, the app categorizes the walk as festination (501) and starts to provide auditory cueing per the patient's preferences by generating the physiotherapist's counting sound via the in-ear headphone which is wirelessly connected to the smartphone.

The patient still goes into freeze (FIG. 5—502), the app detects this and provides the specially recorded message that helps the patient come out of freeze.

The patient continues to walk normally.

### Example 3: Use by a Group of Patients

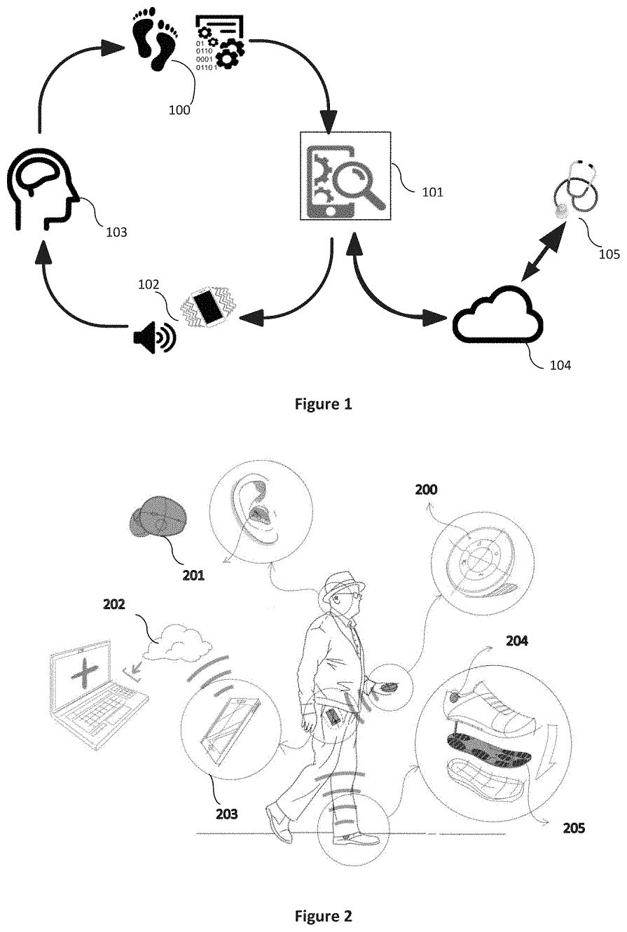

To better exemplify reference is made to FIG. 1, which show(s) the link from the smartphone to the cloud database.

The app in the smartphone transmits the recorded data to the cloud database on a regular basis.

The analysis software looks for similarities across patients in terms of type of normal gait, how and when deviations in gait occur as well as additional patient data such as sex, age, type and dosage of medication and how long they have had the disease for.

Within one such group of patients, the analysis software notes that one patient comes out of freeze much faster than the other patients.

In order to determine the cause of this deviation, the analysis software compares all parameters set by the patients in the group and sees that the audio message for freeze for this patient is very different from the other patients.

The analysis software sends e-mails to the care givers for 3 other patients in the group to request permission to modify the auditory cueing for freeze. 3 other patients in the group have given permission for the analysis software to modify parameters without asking.

2 out of the 3 patients who were asked for permission, give it.

The analysis software now modifies the freeze auditory cue for the 5 patients to be the same as for the one patient with shorter freeze.

Over a 5 week period, the analysis software calculates the freeze duration for the 5 patients and compares that to the other members of the group and the 5 patients before the change was made.

Upon confirmation that the 5 now have reduced freeze duration, the analysis software proceeds to update the optimal settings for this group to include the new freeze auditory cue.

The optimal settings are now communicated to the apps for all patients in the group per their preference (automatic update or update only after patient confirmation).

### Example 4: Use by a Neurologist

A neurologist has a number of patients using the method and/or the system.

Before a visit by one of the patients, the neurologist uses the portal to receive a report about the patient's disease evolution.

The neurologist can see specific indices summarizing the measured data for the patient, such as number of freeze episodes over time, number of festination episodes over time, change in number of freeze episodes relative to changes in medication, number of parameter changes due to group optimization and number of parameter changes due to changes in the patient's walking pattern.

The neurologist can interactively determine which information they need in order to optimize disease treatment and mitigation.

The present invention is in no way limited to the embodiments described in the examples and/or shown in the figures. On the contrary, methods according to the present invention may be realized in many different ways without departing from the scope of the invention.

### CLAUSUS

1. A method for detecting and alleviating symptoms of a deviating gait disorder in a human patient, comprising steps of:
a. continuous registering a person's gait using a sole embedded in the shoes of said patient, said sole comprises at least two pressure sensors in or on the surface of said sole,
b. sending information based on a person's gait from said sole to a receiver device,
c. analyzing and interpreting said information received by said receiver device in order to detect whether said gait is deviating or not,
d. upon detecting a deviating gait, sending single or multiple cues to said patient in order to overcome said deviating gait.

2. Method according to clause 1, wherein the method further comprises any of the following or a combination thereof:
a. a cloud portal giving a care professional access to patient data,
b. a cloud portal analyzing algorithm performance over time and modifying the algorithm in the receiver in order to enhance algorithm performance, and
c. a cloud portal giving a patient or their care givers access to gait performance.

3. Method according to clauses 1 or 2, wherein said pressure sensors detect pressure variations of the foot on said sole when walking.

4. Method according to any of the previous clauses, wherein said sole comprises at least five pressure sensors, preferably located under the heels, under the toes and in metatarsal positions

5. Method according to any of the previous clauses, wherein said information from said pressure sensors is sent to a receiver, wherein said information is analyzed by a computerized algorithm in order to detect whether said gait is deviating or not.

6. Method according to clause 5, wherein said computerized algorithm is stored in the cloud or on said receiver.

7. Method according to any of the previous clauses, wherein one or more cues are sent within 1 second after detecting said deviating gait.

8. Method according to any of the previous clauses, characterized in that said receiver device is a wearable computer device, preferably a smartphone.

9. Method according to any of the previous clauses, characterized in that said cue is an auditory, haptic or visual cue.

10. Method according to any of the previous clauses, characterized in that said cues are sent to a cueing device, said cueing device comprises a headphone, a vibrating device or a light projection device or other visual device.

11. Method according to any of the previous clauses, wherein said cueing device and receiver are present in one device.

12. Method according to any of the previous clauses, wherein said cueing device and receiver are present in multiple, communicating devices.

13. Method according to any of the previous clauses, characterized in that said information based on a person's gait is collected in a database, preferably a cloud database.

14. Method according to any of the previous clauses, characterized in that said human patient suffers from Parkinson's Disease.

15. Method according to any of the previous clauses, characterized in that said symptoms are festinating gait or freezing of gait.

16. Method according to any of the previous clauses, characterized in that said human patient suffers from other neurological diseases such as CVA, MS or polyneuropathy.

17. Method according to any of the previous clauses in that said symptoms are circumduction.

18. Method according to any of the previous clauses, characterized in that said human patient suffers from amputation of the lower limbs.

19. Method according to any of the previous clauses in that said symptoms are phantom pains.

20. A system for detecting and alleviating symptoms of a deviating gait disorder in a human patient, comprising:
a. a sole comprising at least two pressure sensors in or on the surface of said sole for registering a person's gait,
b. a sender sending information based on a person's gait from said sole to a receiver device,
c. a receiver device, for receiving said information,
d. a device for analyzing and interpreting said received information in order to detect whether said gait is deviating or not,
e. a cueing device, sending single or multiple cues to said patient in order to overcome said deviating gait.

21. System according to clause 20, wherein the system further comprises any of the following or a combination thereof:
a. a cloud portal giving a care professional access to patient data,
b. a cloud portal analyzing algorithm performance over time and modifying the algorithm in the receiver in order to enhance algorithm performance, and
c. a cloud portal giving a patient or their care givers access to gait performance.

22. System according to clause 20 or 21, characterized in that said sole further comprises at least one accelerometer, magnetometer or gyroscope.

23. System according to clauses 20-22, wherein said sole comprises at least five pressure sensors, preferably located under the heels, under the toes and in metatarsal positions

24. System according to clauses 20-23, characterized in that said receiver device is a wearable computer device, preferably a smartphone.

25. System according to any of the previous clauses 20-24, characterized in that said cue is an auditory, visual or sensory cue.

26. System according to any of the previous clauses 20-25, wherein said cueing device and receiver are present in one device.

27. Method according to any of the previous clauses 20-26, wherein said cueing device and receiver are present in multiple, communicating devices.

28. System according to any of the previous clauses 20-27, characterized in that said cueing device comprises a headphone, a vibrating device or a light projection device.

29. System according to any of the previous clauses 20-28, characterized in that said portal provides feedback to care professionals.

30. System according to any of the previous clauses 20-29, characterized in that said portal provides feedback the patient and their care givers.

31. System according to any of the previous clauses 20-30, characterized in that said portal improves algorithm performance and cueing effect to optimize patient gait performance.

1-38. (canceled)

39. A method for detecting and overcoming a deviating gait, comprising steps of:
a. continuous registering a person's gait using a sole embedded in shoes of said person, said sole comprises at least two pressure sensors,
b. sending information based on said person's gait from said pressure sensors of said sole to a receiver device,
c. determining that said person's gait is deviating by analyzing said information by a computerized algorithm,
d. providing or sending single or multiple cues to said person in order to overcome said deviating gait.

40. The method according to claim 39, wherein said pressure sensors detect pressure variations of the foot on said sole when walking.

41. The method according to claim 39, wherein the computerized algorithm analyses detailed timing information of said sensors, accumulating or using averages during normal gait and identifying deviations from those averages per area of the foot, such as toes, heels and metatarsals, to determine whether said gait is deviating, said normal gait is based on an average of at least two or three previous steps.

42. The method according to claim 39, wherein said computerized algorithm is stored in the cloud with a cloud based device, or on said receiver device.

43. The method according to claim 39, wherein one or more cues are provided or sent within 1 second after detecting said deviating gait.

44. The method according to claim 39, wherein said information based on a person's gait is collected in a cloud database.

45. A system for detecting and overcoming a deviating gait, comprising:
a. a sole comprising at least two pressure sensors for registering a person's gait,
b. a sender sending information based on a person's gait registered by the pressure sensors from said sole to a receiver device,
c. the receiver device, for receiving said information,
d. a device for analyzing and interpreting said received information in order to detect whether said gait is deviating or not,
e. a cueing device, sending single or multiple cues to said person in order to overcome said deviating gait.

46. The system according to claim 45, wherein the system further comprises any of the following or a combination thereof:
a. a cloud portal analyzing algorithm performance over time and modifying an algorithm in the receiver device or in a cloud-based device communicating with the receiver device, in order to enhance algorithm performance, and
b. a cloud portal giving said person or their care givers access to gait performance.

47. The system according to claim 45, wherein said sole further comprises at least one accelerometer, magnetometer or gyroscope.

48. The system according to claim 45, wherein said sole comprises at least five pressure sensors, located under the heels, under the toes and in metatarsal positions

49. The system according to claim 45, wherein said cueing device is configured to provide an auditory, visual and/or sensory cue.

50. The system according to claim 45, wherein said device and receiver device are present in one device or said device is a cloud-based device.

51. The system according to claim 45, wherein said cueing device and receiver device are present in multiple, communicating devices, the sole comprising the pressure sensors is another device communicating in a wireless manner with the cueing device by means of the receiver device.

52. The system according to claim 45, wherein said cueing device comprises a headphone, a light projection device and/or a vibrating device, the vibrating device is releasably attached by a strap or the like to the person's upper body around or above the person's waist such as around an arm.

53. The system according to claim 46, wherein said cloud portal uses cloud software for analyzing algorithm and cueing performance to improve algorithm performance of the device and the effects of the cueing in terms of bringing the deviation back to zero to optimize gait performance.

54. The system according to claim 45, wherein the device is configured to analyze heel down and toe off pressures to detect whether gait is deviating or not and/or in which way it is deviating.

55. The system according to claim 45, wherein after providing the single cue or multiple cues, the device is configured to monitor in real time the person's gait for determining a deviating gait reduction,
wherein by determining in real time that the deviating gait reduction is below a threshold a second single cue or second multiple cues is/are provided,
wherein the previous single cue or previous multiple cues is/are different than the second single cue or second multiple cues, by a different type of cuing, different duration of cuing, different frequency of cueing, different volume of cuing, different tactile/visual experience of cuing or different auditory instructions of cuing or by using two cueing devices simultaneously.

56. The system according to claim 45, wherein the device is configured to analyze said information by comparing said information with a normal gait pressure pattern.

57. The system according to claim 56, wherein the device is configured to base said normal gait pressure pattern on pressure patterns of at least two or three previous steps of the registered person's gait.

58. The system according to claim 57, wherein the device is configured to base said normal gait pressure pattern on an average of at least two or three previous steps of the registered person's gait.
